(19) United States
(12) Patent Application Publication
(10) Pub. No.: US 2004/0167415 A1
(43) Pub. Date: Aug. 26, 2004

(54) Method and system for prevention of radiocontrast nephropathy

(75) Inventors: Mark Gelfand, New York, NY (US); Howard R. Levin, Teaneck, NJ (US)

Correspondence Address: NIXON & VANDERHYE, PC, 1100 N GLEBE ROAD, 8TH FLOOR, ARLINGTON, VA, 22201-4714, US

(73) Assignee: PLC Systems Inc., Franklin, MA (US)

(21) Appl. No.: 10/784,231

(22) Filed: Feb. 24, 2004

Related U.S. Application Data
(60) Provisional application No. 60/449,174, filed on Feb. 24, 2003.
(60) Provisional application No. 60/449,263, filed on Feb. 24, 2003.

(51) Int. Cl. A61B 5/02

(52) U.S. Cl. 600/500

(57) ABSTRACT

A method for protecting a kidney in a mammalian patient from an insult including the steps of: at least partially occluding at least one renal vein of the patient; elevating a renal vein blood pressure, and reducing the renal vein blood pressure from the elevated blood pressure

### RELATED APPLICATION

This continuation application claims priority to and incorporates by reference U.S. Provisional Application Serial No. 60/449,174, filed Feb. 24, 2003, and U.S. Provisional Application Serial No. 60/449,263, also filed Feb. 24, 2003

### FIELD OF THE INVENTION

This invention relates to a method for preventing radiocontrast associated nephropathy and protection of human kidneys from failure due to a radiocontrast solution. The invention also relates to a renal vein or ureter occlusion catheter.

### BACKGROUND OF THE INVENTION

Intravascular iodinated radiocontrast solution (further called contrast or radiocontrast for simplicity) is opaque to x-rays and enables the circulatory system arteries and veins to be visualized. Iodinated contrast is used in medical procedures such as diagnostic angiography, percutaneous transluminal coronary angioplasty (PTCA), peripheral vessel studies and interventions and placement of pacemaker leads.

From the visualization point of view, there are three phases of intravascular contrast enhancement: bolus or arterial phase, nonequilibrium or venous phase, and the equilibrium or portal phase. The bolus phase represents the critical time of peak enhancement within the target vessel or organ and occurs immediately after the injection of contrast, and lasts between 10 seconds and 60 seconds postinfusion depending on the amount and site of injection. For coronary angiography, the visual opacification by injection of a 5 cc bolus of a radiocontrast agent solution into the coronary artery will last much shorter than a 70 cc bolus injected into the left ventricle. The nonequilibrium phase occurs approximately 1 minute after the bolus of contrast media. The bolus of contrast is injected 109 into the vein of a patient. The last phase is considered the equilibrium phase, which occurs approximately 2 minutes after the bolus injection. Thus, the contrast agent becomes equally distributed in the total blood (plasma) volume by about 2 minutes after a single injection.

Common currently used contrast agents consist of iodinated benzene ring derivatives. The multiple iodine molecules contained within the contrast agent are responsible for additional attenuation of X-rays in excess of that caused by the blood alone. In clinical practice, the attenuation of the X-rays by injection into a blood vessel of the iodinated contrast agents in the bolus phase is of sufficient magnitude for the blood vessel to appear markedly more opaque than the adjacent areas without contrast material. The amount of radiopacity that is generated by a particular contrast agent is a function of the percentage of iodine in the molecule and the concentration of the contrast media administered. The iodine content in different radiographic contrast media can vary from 11% to 48%. With most contrast solutions the iodine content is also proportional to the osmolarity of the contrast agent. Iodinated contrast agents are classified as ionic, high osmolar contrast media, nonionic or low osmolar contrast media. The osmolarity of the contrast agent can lead to significant side effects in clinical practice. In general, the lower the osmolarity of the agent, the less side effects will occur in the patient.

The use of the contrast solution, now ubiquitous in modern medicine, still includes a certain amount of risk. Even with the use of the most advanced, non-ionic compounds, which are inert and hypoallergenic, contrast associated nephropathy (damage to the kidneys) remains a significant, unsolved clinical problem.

Renal dysfunction has been long recognized to be associated with the use of radiographic contrast media. Ideally, renal function is determined by the measurement of glomerular filtration rate (GFR). However, the methods of measurement of GFR are cumbersome, lengthy and generally not applicable to many clinical situations. In common clinical practice, the GFR is estimated by measurement of the serum creatinine, a molecule in the blood whose concentration is primarily dependent on the kidney for removal.

The spectrum of renal dysfunction ranges from a transient slight increase in serum creatinine levels to overt renal failure requiring transient or long-term dialysis. Mild, transient decreases in renal function occur after contrast administration in almost all patients. Whether a patient develops clinically significant acute renal failure, however, depends very much on the presence or absence of certain risk factors. Baseline renal impairment, diabetes mellitus, congestive heart failure, and higher doses of contrast media increase the risk of contrast nephropathy (CN). Other risk factors include reduced effective arterial volume (e.g., due to dehydration, nephrosis, cirrhosis) or concurrent use of potentially nephrotoxic drugs such as nonsteroidal anti-inflammatory agents and angiotensin-converting enzyme inhibitors. Of all these risk factors, preexisting renal impairment appears to be the single most important. Patients with diabetes mellitus and renal impairment have a substantially higher risk of CN than patients with renal impairment alone.

Though many different definitions of CN appear in the literature, it can be defined in general as an acute decline in renal function following the administration of intravenous contrast in the absence of other causes. Contrast nephropathy is commonly defined clinically as a rise of 0.5 mg/dl, or a rise of 25% or more from the patient's baseline creatinine. Patients with CN typically present with an acute rise in serum creatinine anywhere from 24 to 48 hours after the contrast study. Serum creatinine generally peaks at 3 to 5 days and returns to baseline value by 7 to 10 days.

Prospective studies have produced varied estimates of the incidence of CN. These discrepancies are due to differences in the definition of renal failure as well as differences in patient comorbidity and the presence of other potential causes of acute renal failure. A recent epidemiological study reported a rate of 14.5% in a series of approximately 1800 consecutive patients undergoing invasive cardiac procedures. Patients without any significant risk factors have a much lower risk, averaging about 3% in prospective studies. On the other hand, the risk of renal failure after contrast rises with the number of risk factors present. In one study, the frequency of renal failure rose progressively from 1.2 to 100% as the number of risk factors went from zero to four.

Accordingly, the problems associated with contrast nephropathy have been a limiting factor on the extent to which these advanced angioplasty procedures can be used particularly in vulnerable patient populations.

There is a long recognized need to reduce the incidence and severity of contrast associated nephropathy caused by iodine containing contrast that is toxic to kidneys. Multiple studies have established a correlation between the extent of kidney damage caused by contrast injections and the amount of contrast administered during the intervention. It clinical practice, when treating a vulnerable patient, interventionalists tend to use less contrast at one time and space procedures, otherwise performed sequentially, between several sessions, even over several days. Thus, it is clear that vulnerable patients may, at best, have significant delays in completing potentially life saving treatments (such as coronary angiography, CAT scans or coronary artery bypass surgery), markedly increasing the risk of further complications or death. In the worst cases, the patients may totally be denied access to these life-saving treatments. Methods are needed that mitigate the potentially deleterious effects of the contrast agents and allows more rapid, consistent access to these life-saving therapies.

In a conventional procedure, after an injection, contrast is cleared (removed) from the body solely by the kidneys. In the kidneys, contrast is cleared by passive filtration or convective transport in the tubules. The glomerular filtration rate (GFR) of a kidney is essentially equal to the rate at which blood is cleared of the contrast. For example if a kidney filters 65 ml/min of blood, the same amount of blood is cleared of contrast per minute by one kidney. Molecules of contrast are dragged by the flow of filtrate across the glomerular membrane of the kidney with water and other small molecules from plasma. Most of the water is immediately reabsorbed back into the kidney but the contrast is collected in the tubules of the kidney and removed with urine.

Any drug has a “therapeutic window”. The therapeutic window is the range of drug concentration in the blood where one expects to see the desired clinical effect of the drug with the minimal amount of side effects. If the concentration is below the therapeutic window, the beneficial effects are minimal or non-existent. If the concentration is above the therapeutic window, the side effects become very prominent.

Drugs come in different dosages. Certain characteristics of patients (such as size, amount of excess fluid in the body, total fat content, ability to absorb the drug in the stomach or intestinal tract) affect the blood level achieved by a given dosage of a drug. Physicians must individualize the dose of each drug to compensate for these characteristics to achieve a blood level within the therapeutic window.

Like any other drug, contrast agents affect the target organ in proportion to the concentration of the active chemical agent (in this case iodine) in blood plasma that flows through the organ. In addition, the duration of the exposure to the agent is another key parameter that defines the end effect and potential damage to the organ. The concentration of contrast is, at any given time after the injection, equal to the amount of contrast that was injected minus the amount cleared by kidneys divided by the volume of distribution.

The total volume of distribution of a typical contrast agent, iohexol, is according to the manufacturer Nycomed Amersham approximately 18 liters in a 70 kg adult patient. After a bolus injection, the contrast agent is almost immediately mixed into the approximately 3 liters of blood plasma. The contrast concentration in blood is maximum at this point. Over time, the concentration of contrast in the blood is reduced as the contrast is redistributed into the total volume of extracellular water in the body tissues. The exact way in which contrast is redistributed and cleared from the body is very similar to any other drug and follows the equations well described in the field of pharmacokinetics. Time constants that allow fairly accurate reconstruction of the concentration (in blood or plasma) vs. time curves for frequently used contrast agents is available from manufactures such as Nycomed Amersham as a public record required by the Food and Drug Administration. Generally, after the injection, contrast concentration follows the exponential decay curve known as the first order kinetics.

The parameters of the pharmacokinetic model generic to all drugs, such as contrast agents, influence the maximum (peak) concentration, the time at which the maximum concentration occurs (peak time), and the area under the concentration-time curve after a single intravascular injection dose. Although the exact parameters for any individual drug can vary depending on the permeability of membranes to that specific drug separating various compartments of the distribution volume, the general principles remain the same.

Pharmacokinetics of various contrast injections is well studied in humans. Two and three compartment models of contrast distribution models produced good fit to experimental concentration-time curves. Regardless of the particular model and parameter set used, it is established that the contrast concentration in blood peaks sharply immediately after a single injection. The peak concentration is followed by the relatively fast exponential decrease of concentration over the following 20-60 min while the contrast is redistributed in the much larger extracellular fluid volume than the initial volume of blood plasma. This phase is followed by the slow phase of elimination while contrast is removed from blood by kidneys. On average, it can take up to 12-24 hours to remove most of the injected contrast from a normal person.

During a medical intervention, such as angiography, contrast is given in a series of bolus injections typically into a coronary artery of the patient. While each bolus is small (5-15 ml), a total of as much as 150-300 ml of contrast can be infused during the procedure. Since the total time of the procedure rarely exceeds one hour, the contrast concentration in blood increases with each injection. Rapid injections do not allow sufficient time for the contrast to redistribute from the blood into the total extracellular body water distribution volume. As a result, the concentration of contrast in blood keeps increasing and can peak at dangerously high levels, well outside of its therapeutic window. Even healthy kidneys require many hours to eliminate contrast from blood. Renal clearance itself has little immediate effect on contrast removal and does not effect the peak contrast concentration in blood. For example in “Pharmacokinetics of Iohexol, a New Nonionic Radiocontrast Agent, in Humans” (J Pharm Sci 1984 July; 73 (7): 993-5) Edelson et al established that 90% of contrast was eliminated from the body in urine in 12 hours by kidneys in healthy people.

Based on the known pharmacokinetics confirmed by clinical studies it is clear that kidneys are exposed to relatively high concentration of contrast in blood during the time window that corresponds to the peak concentration of contrast. Depending on the sequence of injection during the medical procedure and parameters of the pharmacokinetic model, this peak concentration window can last approximately 30 minutes to 2 hours. At the end of this period, the concentration of contrast that passes through kidneys with blood flow can be 5 to 10 times lower than at the time of the peek.

It is reasonable to conclude (from the known physiology of contrast induced nephropathy and renal failure) that, in standard clinical practice using contrast agents, the kidneys are damaged primarily by exposure to high concentrations of contrast in blood. As a general rule, kidneys can continuously excrete low concentrations of various drugs or toxins over time as a part of their normal function without sustaining damage. However, exposure to high concentrations of the same toxin, even over a short period of time, can lead to the significant and lasting damage.

The threshold or exact concentration at which renal damage by a contrast agent will occur (e.g., the top level of the therapeutic window for each contrast agent) is not known and is likely to be different for different patients. It is believed that if less that 50 ml of contrast is injected during a procedure the kidneys are almost never damaged. At the same time, it is known clinically that in procedures involving the use of 150 or more ml of contrast, the risk of contrast nephropathy (renal damage from the contrast) becomes increasingly high.

Regardless of the exact mechanism of contrast nephropathy, it is clinically accepted and physiologically reasonable to believe that the reduction of exposure of a kidney to high peak concentration of contrast agents in blood will be beneficial, especially in vulnerable patients such as diabetics.

### SUMMARY OF THE INVENTION

A novel and unobvious method and system has been developed to reduce the exposure of at least one kidney to high concentrations of contrast agents in blood in a patient undergoing a procedure that involves intravenous injections of contrast. The contrast may constitute an insult to the kidney that can (if untreated) harm the kidney. Similarly, other potential insults to the kidney are some surgical procedures and hypotension. In a general sense, the method and system disclosed here can be applied to reduce the exposure of one or both kidneys to insults such as contrast injections, surgical procedures and hypotension.

It is established that the high concentration duration (also called time period or time window) can last up to several hours until the contrast is sufficiently redistributed into the total body extracellular fluid volume. The total body extracellular fluid volume can be as much as 10 times larger that the volume of blood plasma in which the contrast agent is initially diluted. Accordingly, after the redistribution, the concentration of the contrast agent in the blood is 10 times lower and significantly less hazardous to the kidney. After the contrast concentration is sufficiently reduced by redistribution of the contrast molecules into the total extracellular fluid volume, the therapy can be stopped.

The method and system temporarily reduce the flow of blood that passes through at least one kidney (renal perfusion) and the flow of filtrate that is extracted from blood inside the kidney (GFR) for the duration of the peak concentration window. In the “Effect of Increased Renal Venous Pressure On Renal Function” (Journal of Trauma: Injury, Infection and Critical Care 1999, Dec; 47(6): 1000-3) Doty et al describe effects of elevated pressure in the renal vein on the blood flow and GFR of the kidney. Doty concluded that in the experimental 20 kg pigs, elevation of renal venous pressure (RVP) to 0-30 mm Hg above baseline resulted in the significant decrease in renal artery blood flow index from 2.7 to 1.5 mL/min per gram and glomerular filtration rate from 26 to 8 mL/min compared with control. Importantly, these changes were partially or completely reversible as RVP returned toward baseline.

Similar conclusions can be reached by studying clinical experience with the disease known as an acute abdominal compartment syndrome. Patients with compartment syndrome often have elevated renal vein blood pressure due to partial occlusion or compression of the renal vein. It was observed in patients with the renal vein pressure elevated by 30 to 60 mmHg over a baseline pressure the kidneys stopped making urine but generally were not permanently damaged. Renal function is promptly restored in these patients when the surgeon relieves the abdominal compression and hence the renal vein pressure. In patients that, as a result of the compartment syndrome, had renal vein pressure elevations of more than 60 mmHg, the kidneys were often damaged temporarily or even permanently.

In normal humans, baseline renal vein pressure is between 0-5 mmHg. Patients with right side heart failure and chronically elevated venous pressure of 20-30 mmHg often exhibit diminished renal function and reduced renal blood flow. However, even if the exposure to this increased pressure is prolonged over weeks or months, the renal function is known to improve when the renal vein pressure is reduced and as long as the renal vein pressure did not exceed 60 mmHg.

Based on the physiologic response of the kidney to the elevated renal vein blood pressure, a counterintuitive method and system have been developed to protect kidneys from contrast nephropathy. In one embodiment, the method reduces perfusion and GFR of at least one kidney temporarily to reduce the exposure of the kidney to the high concentration of contrast.

In one embodiment, the method and system comprises temporarily increasing renal vein pressure by creating a removable obstruction of the renal vein. The obstruction is controllable so that it creates the renal artery backup pressure of 30 to 60 mmHg by partially obstructing but not totally blocking the renal vein outflow. Within the scope of this application, the words occluding, blocking and obstructing have the same meaning when applied to a body fluid passage.

### SUMMARY OF THE DRAWINGS

A preferred embodiment and best mode of the invention is illustrated in the attached drawings that are described as follows:

FIG. 1 is a schematic diagram of the kidneys and vascular system in a patient to illustrate the treatment of contrast nephropathy with a partially occluding balloon in the renal vein.

FIG. 2 illustrates the placement of the renal vein catheter in a patient.

FIG. 3 illustrates an apparatus for partially occluding a renal vein.

FIG. 4 is a schematic diagram of a distal tip of a renal catheter, showing the catheter partially in cross-section.

FIG. 5 is an end view of a cross-section of the distal tip of the catheter.

FIG. 6 is a time-concentration curve for intravenous use of radiocontrast.

FIG. 7 is a flow chart for an exemplary control algorithm for balloon inflation of the catheter.

FIG. 8 is a pair of graphs illustrating the effect of the balloon inflation on the renal vein pressure.

FIG. 9 is a schematic diagram of the kidneys of a patient and a renal pelvis pressure embodiment of the subject invention.

### DETAILED DESCRIPTION OF THE INVENTION

For the proposed clinical use, the method and system disclosed herein protects a kidney of a patient from nephropathy caused by the intravenous injection of radiocontrast media. It is understood that the same or similar method and apparatus can be used to protect the kidney from other toxic substances. It is also understood that other embodiments that achieve substantially the same goal of temporarily reducing blood perfusion and GFR of at least one kidney are within the scope of the method and system. Common to these embodiments is that blood or urine pressure downstream of the kidney is increased above normal but below the level that can cause injury to the kidney.

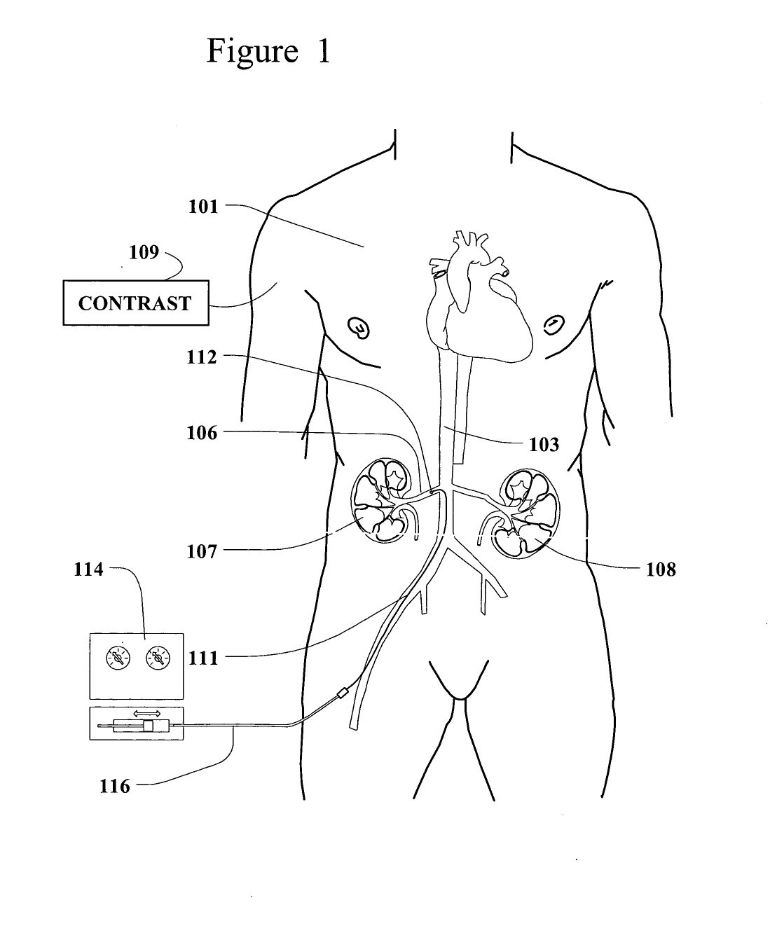

FIG. 1 illustrates the treatment of a patient 101 to protect the kidney 107 from contrast nephropathy. The device basically consists of the vascular catheter 111, inflatable balloon 112 on the distal (remote, farther from the operator) end of the catheter and the balloon inflation controller 114 connected to the proximal (nearest or closer to the operator) end of the catheter. Other elements of the device are not shown on this high level drawing.

The catheter 111 is inserted into the femoral vein of the patient from an incision or puncture in the groin area. The catheter has outer diameter of up to 9 French but preferably 5 French or less. The catheter is advanced downstream (towards the heart) first into the femoral vein and further into the inferior vena cava (IVC) 103. During the insertion of the catheter, the balloon 112 is deflated and collapsed so as not to interfere with the blood flow and to allow passage through small openings and vessels. Using common fluoroscopic or ultrasonic navigation and interventional tools such as guide wires and guiding catheter sheaths, the distal tip of the catheter 111 is inserted into the renal vein 106. The first objective of the treatment is to position the balloon in the renal vein and to inflate it there.

The renal vein in humans is approximately 8 to 12 mm in diameter at the junction to the IVC. Therefore, when inflated, the balloon 112 shall expand to the diameter of approximately 5 to 8 cm to effectively partially occlude the renal vein 106. This partial occlusion creates resistance to blood flow draining from the kidney 107 towards the IVC 103. As a result of this increased resistance, pressure in the renal vein segment between the kidney and the balloon (upstream renal vein pressure) is elevated. Pressure below the balloon (downstream renal vein pressure) is approximately equal to the IVC pressure.

The contralateral kidney 108 may not be protected. It is assumed that it will make urine and clear the contrast during the procedure. If it is damaged, it is likely to recover on its own over time while the protected kidney 107 performs normal renal functions. In an alternative embodiment, both kidneys can be protected in the same way. In the end, it is likely to be a clinical decision made by the physician rather than an aspect of technology.

The proximal end of the catheter 111 is attached to the control and monitoring console 114 by a flexible conduit 116. The conduit 116 can include a balloon inflation lumen and signal-conducting means for pressure measurement. The console 114 includes a microprocessor with embedded software code as well as the sensors and actuators needed to monitor pressures and control the inflation and deflation of the balloon 112.

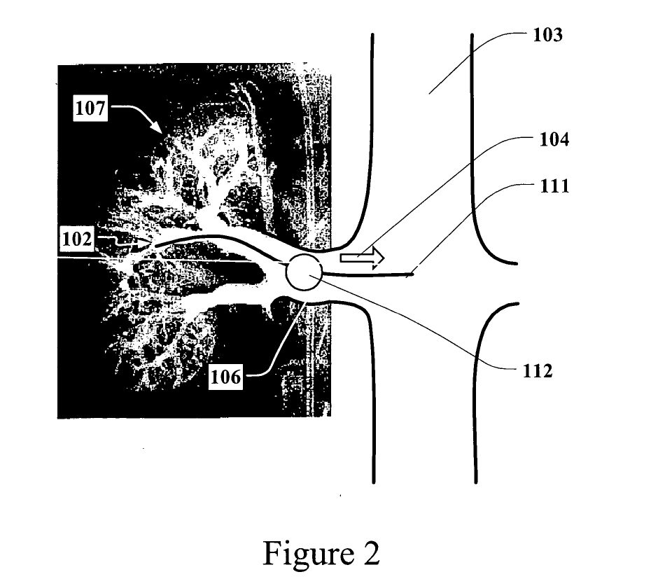

FIG. 2 further illustrates the distal catheter end and balloon position in the renal vein 106 of the kidney 107 using a renal venogram (contrast enhanced X-ray image). The balloon 112 partially occludes the renal vein thus impeding flow of blood from the kidney veins into IVC 103. The distal catheter tip 102 deeply penetrates into one of the smaller veins of the kidney to prevent migration of the balloon into IVC with the venous blood flow 104. It is understood that other ways to anchor the catheter in place can be designed by an experienced catheter engineer. The balloon 112 is positioned near the junction of the renal vein 106 and the IVC 103. The balloon can partially or completely reside in the IVC and efficiently impede the outflow of blood from the junction. Alternatively, it can be used to occlude or partially occlude larger branches of the renal vein tree and achieve the same effect of increasing renal venous pressure. It is understood that the catheter based devices to partially occlude a blood vessel other than inflatable balloons can be used to implement the invention. For example U.S. Pat. No. 6,231,551 “Partial Aortic Occlusion Devices and Methods For Cerebral Perfusion Augmentation” describes a mechanical occlusion device that can be adapted for this invention. The balloon catheter is chosen for the preferred embodiment because of its simplicity and extensive experience of clinicians who work with balloon-tipped catheters inside the human vascular system.

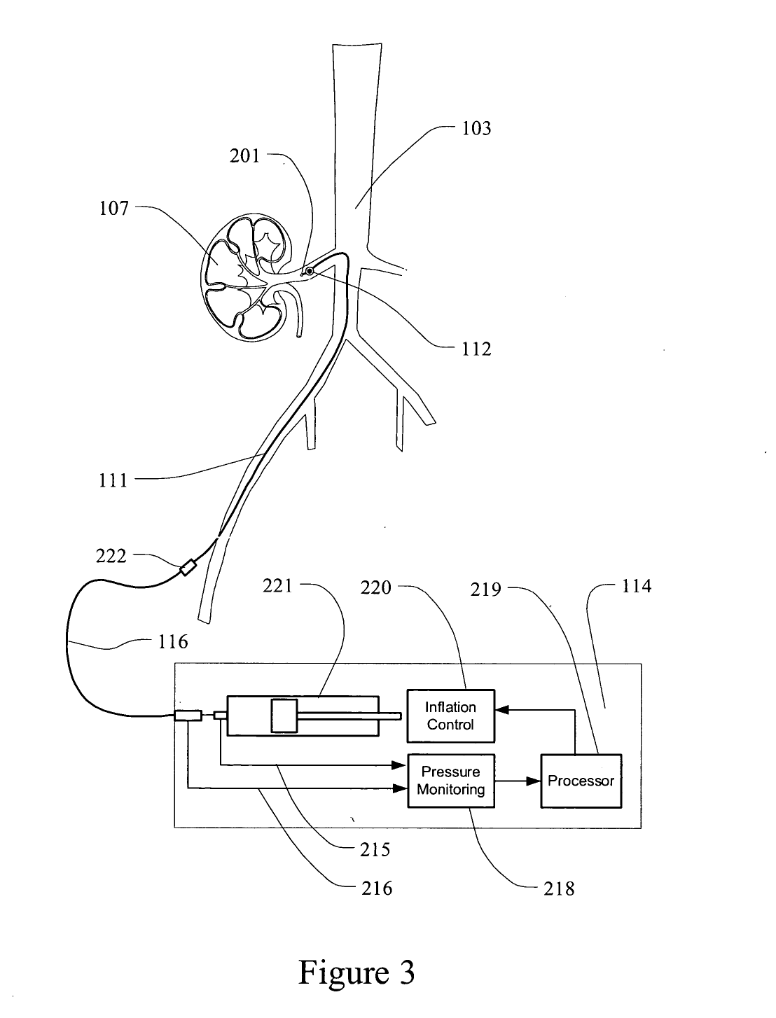

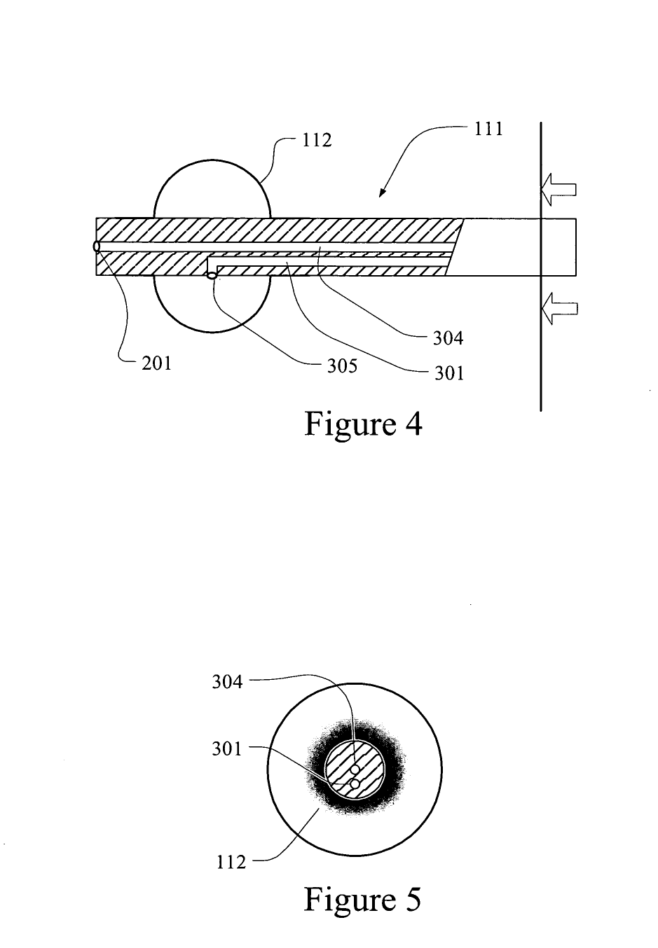

FIG. 3 shows an embodiment of the partial renal vein occlusion apparatus in more detail. The catheter 111 is positioned in the IVC 103 with the partially occluding balloon 112 located in the renal vein upstream of the renal vein-IVC junction and downstream of the kidney 107. The distal end of the catheter 111 is equipped with a balloon 112. The proximal end of the catheter 111 is connected to the flexible conduit 116 with the coupling device 222. The conduit 116 connects the catheter 111 with the controller device 114. The catheter is equipped with at least one pressure measurement lumen (see FIGS. 4 and 5) that terminates in the distal opening 201. The pressure measurement lumen is connected to the pressure monitoring part 218 of the controller 114 via the conduit 116.

The controller 114 includes the balloon inflation device 221, such as a syringe pump that operates as a piston. Merit Medical Inc. (South Jordan, Utah) offers a wide variety of these type inflation devices of balloon tipped catheters that can be easily adopted for the apparatus. For example, Merit Medical manufactures an IntelliSystem Inflation Syringe for balloon catheters used in interventional cardiology to inflate angioplasty balloons inside the coronary arteries of the heart.

Alternatively, other devices commonly used to inflate catheter balloons with compressed gas can be used. For example, a cylinder with compressed gas under high pressure (not shown) can be connected to the catheter 111 using a pressure regulator and a control valve. The inflation gas can be air, helium or carbon dioxide. Alternatively, the balloon 112 can be filled with liquid such as saline or water. Inflation and deflation of the balloon 112 by the inflation device is controlled by the inflation control electronics 220. The inflation control 220 can include valves, motors and standard motor control electronic devices.

The controller 114 also includes a pressure monitoring system 218. Two pressure measurements may be made of balloon inflation pressure signal on line 215 and of the upstream (distal) renal vein pressure signal on line 216 corresponding to the catheter tip openings 201. The pressure measurement system is in fluid communication with the opening 201 for the purpose of continuous blood pressure measurement. Pressure signals from the pressure monitoring system 218 are transmitted to the processor 219 that in turn controls the inflation of the balloon 212 with the inflation control system 220. The processor 219 includes imbedded software code that is responsible for reading and converting data from pressure sensors and inflation and deflation of the balloon using a real-time control loop.

The pressure monitoring system uses fluid filled tubes to measure blood pressure. Fluid filled tubes are connected to pressure sensors that reside outside of the patient's body. Equipment for this kind of blood pressure measurement is widely available and often used in intensive care units to monitor blood pressure in veins and arteries. Alternatively, more advanced micro-tip pressure transducers (such as the ones manufactured by Millar Instruments Inc. Houston, Tex.) can be integrated with the catheter 111 to obtain more reliable and accurate measurements.

A Canadian company Angiometrx (Vancouver, BC) manufactures the brand name product called Metricath System for sizing blood vessels before stent placement. The Metricath system consists of the inflation console and a balloon tipped catheter. The inflation console is capable of gently inflating the balloon inside the patient's coronary artery until the balloon comes in contact with the arterial wall. The volume of gas used for inflation is measured precisely and the caliber of the vessel is automatically calculated. This example shows that a device for very precise inflation of a balloon inside a human blood vessel can be made using known and available technology.

FIGS. 4 and 5 show two orthogonal cross-sections of the distal end of the catheter 111. The catheter shaft is a tube with two lumens (internal channels) 301, 304. The balloon inflation lumen 301 terminates in the opening 305 inside the balloon 112. The lumen 301 is in fluid communication with the inflation device 221 (See FIG. 3). It is used to inflate and deflate the balloon 212. The pressure measurement lumen 304 terminates in the distal opening 201. The lumen 304 is in fluid communication with the pressure monitoring system 218 (See FIG. 3). This lumen is used to monitor pressure in the renal vein upstream of the balloon that determines the effectiveness of the partial renal vein occlusion therapy.

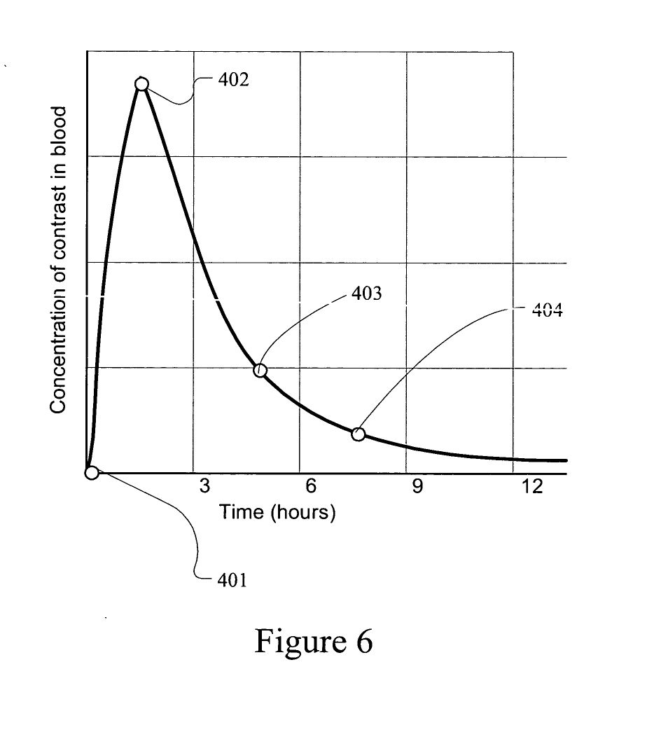

FIG. 6 is a graph that illustrates the changes in the concentration of the contrast in the patient's blood during and after an interventional procedure using a concentration-time curve. The contrast concentration is plotted on the Y-axis in arbitrary units. The first injection of contrast is given to the patient at the point 401 at the beginning of the procedure. The concentration curve starts to rise quickly. The first injection may be commonly followed by many more sequential injections. The concentration of contrast in the plasma rises faster than the redistribution of contrast into the total extracellular body fluid volume or the clearance of contrast from the blood by the kidneys. The contrast injections are stopped at a point 402 that can be 30 minutes to 1.5 hour after the procedure started. The concentration of contrast reached its peak at this point. Depending on the contrast agent used and the nature of the procedure, the contrast concentration at that point can be as high as 4 to 8 gram of Iodine per liter of plasma.

After the contrast concentration has reached its peak 402 and the injections of additional contrast stop, the concentration curve enters into the rapid decline segment between points 402 and 403. The contrast concentration in plasma declines rapidly because it gets redistributed from the vascular compartment (3 liters of plasma) to the total extracellular fluid volume of distribution (20 liters of body water). The contrast concentration at the end of the redistribution period can be 50% to 80% lower than the peek concentration 402 depending on the renal function and the body size of the patient. The rate of decline of the concentration curve slows down between the points 403 and 404, illustrating that the distribution volume typically consists of more than one compartment. Small molecules such as contrast are rapidly redistributed from vascular space to the internal organs such as liver, spleen, lungs and gut. This fast redistribution is followed by the slower phase during which contrast is redistributed into muscle tissues. After the redistribution phase 402 to 404 is complete, the contrast concentration in blood is reduced much slower. During this phase, the kidneys alone clear the contrast from blood. As the concentration of contrast in blood drops, a gradient is now created for movement of contrast from the extracelluar fluid volume back into the blood. As more contrast is recruited from the extracelluar fluid volume into the blood, this contrast is now available for the kidney to remove. The exchange between the body compartments occurs solely by diffusion of contrast molecules across the body membranes.

The method and system disclosed herein protects at least one kidney of the patient from the exposure to high concentration of contrast in blood. This protection is implemented during the rise phase of the contrast concentration-time curve 401 to 402, peak phase (around point 402) and the redistribution phase (403 to 404). Balloon protection can be activated in the renal vein at the beginning of the procedure 401 or shortly thereafter and terminated at the end of rapid (403) or slow (404) redistribution phase of the curve. It is assumed that from the point 404 onward kidneys can clear contrast from blood in low concentration without any damage.

As previously stated, the kidneys can remove many toxins and drugs without causing damage to the kidneys if the concentration of these substances is appropriately low. Since the concentration of contrast in the blood (resulting from the recruitment of contrast back into the blood) remains sufficiently low, the kidneys can removed the total amount of contrast injected over a prolonged period of time without damage. This period of time is commonly 12-24 hours.

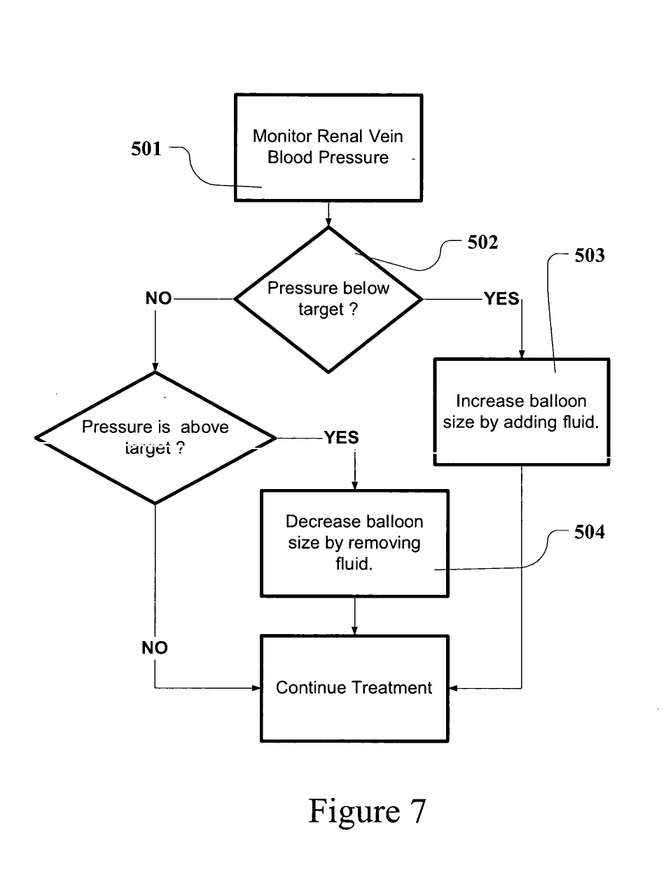

FIG. 7 exemplifies an algorithm that can be embedded in the software of the controller processor 219, FIG. 3. Renal vein pressure is monitored 501 continuously using a pressure sensor (not shown), an amplifier and an analog-to-digital converter. These are the standard components of a conventional and well-known digital pressure monitor that need not be explained in detail. The processor is equipped with an internal clock. Information in digital form is supplied to the processor every 5-10 milliseconds. The software algorithm compares the pressures to the target values set by the operator 502 or calculated by the processor based on other physiologic information such as blood pH or oxygen content. The algorithm commands the inflation 503 or deflation 504 of the balloon 112 (FIG. 3) based on the pressure feedback 501 with the objective of achieving the desired pressure target. Generally the goal of the algorithm is to achieve mean renal venous pressure that is greater than 20 mmHg and less than 60 mmHg.

Implementation in software of the algorithm illustrated by FIG. 7 in the processor 219 can be easily achieved by applying methods known in the field of controls engineering. For example, classic process control algorithms such as a Proportional Integral (PI) controller can be used to maintain pressure at the target level. Control signals can be applied continuously or periodically to adjust the size of the balloon. It can be expected that during the time of the procedure the balloon can stretch, leak gas or that the patient's condition such as the cardiac output and peripheral vascular resistance can change. In response to these changes the renal venous pressure may change requiring the correction. It can be envisioned that the correction will be made by the operator based on the readings of pressure manometers sensing renal pressure via distal outlet 201 but it is preferred to have an automatic response to save time and increase safety.

In addition to the basic control algorithm illustrated by FIG. 7, physiologic data other than blood pressure can be used to guide the therapy. For example, the acidity of blood can be measured using a standard clinical pH monitoring device. An increase of acidity indicates anaerobic metabolism resulting in the production of lactate. It is particularly advantageous to monitor pH of the venous blood returning from the kidney to the central venous blood pool. A drop in pH below preset level or by preset amount can be used to decrease the pressure target 502 since it indicates inadequate perfusion of the kidney and ischemia. Similarly, monitoring of venous blood oxygen content can be used to monitor the same condition. Decrease of oxygen concentration or saturation in renal vein blood will indicate inadequate perfusion or ischemia of the kidney. In addition, central venous pressure can be measured in the IVC to use as a correction to the renal vein pressure target. These measurements and the corresponding equipment are well known in the practice of medicine and are not described in further detail.

FIG. 7 is a pair of panels of charts and graphs that illustrates the effect of the proposed treatment on the blood pressure in the renal vein of the patient. The panel 610 shows the catheter 111 in the renal artery 106 with the balloon 112 inflated. The blood pressure graph below shows the blood pressure measured along the cannulated segment of the renal artery 106. Distally (upstream) of the balloon, 112 the renal vein blood pressure 601 is 25 mmHg, and downstream of the balloon 112, the blood pressure 602 is 5 mmHg (normal venous pressure or the baseline). The following panel 611 shows the same segment of the renal vein with the balloon 112 inflated more. Since the balloon now occludes more of the cross-section of the renal vein, the upstream pressure 603 is now 35 mmHg. The downstream pressure 602 stays 5 mmHg unaffected by the balloon inflation.

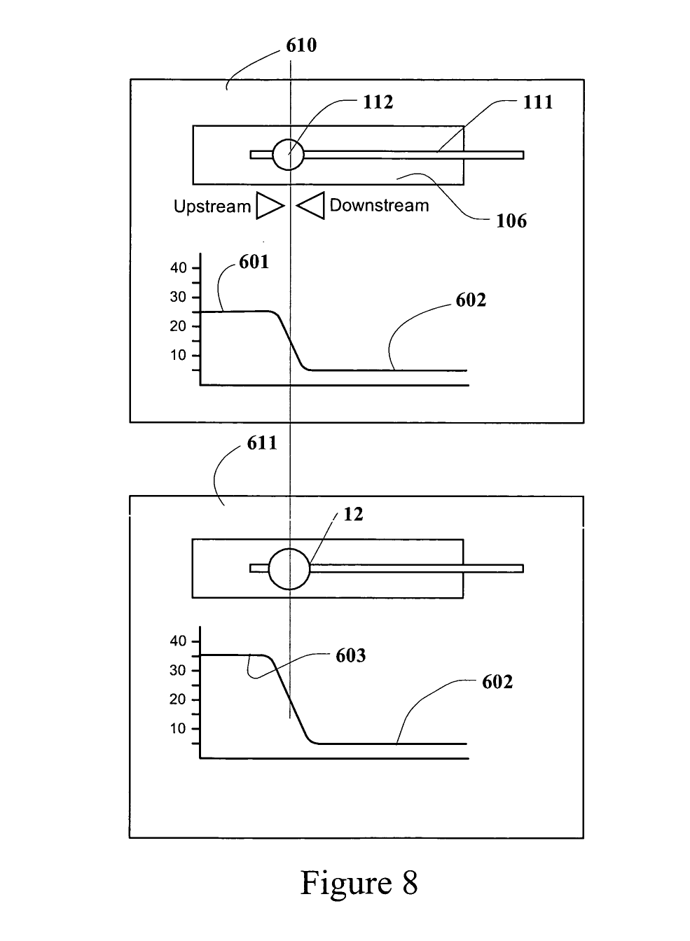

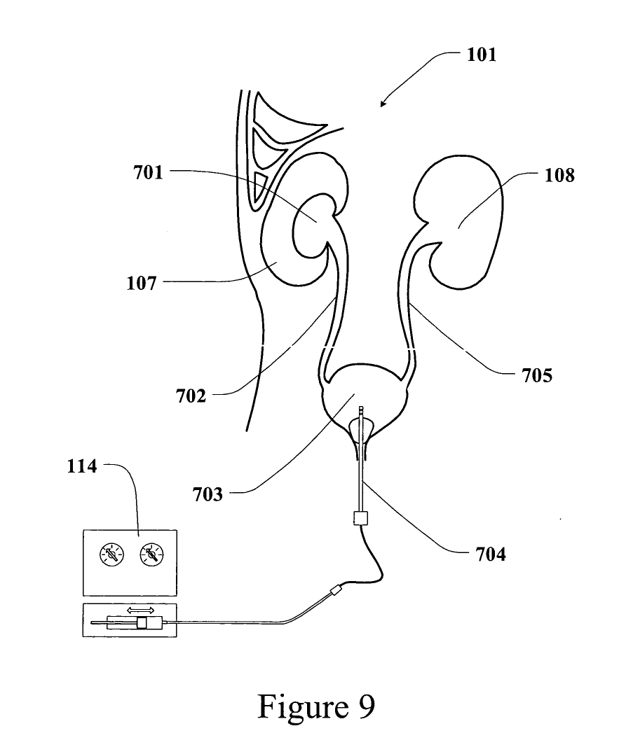

FIG. 8 illustrates an alternative embodiment in which the kidney 701 is protected from contrast nephropathy by temporarily elevating the pressure in the renal pelvis of the kidney 701. The renal pelvis is a cavity in the middle of the kidney that is an extension of the ureter 702. The urine formed in the nephrons of the kidney drains into the renal pelvis. From the pelvis, it drains into the bladder 703 via the ureter 702 and 705. In a normal subject patient, the pressure in the pelvis of the kidney is at the atmospheric level or slightly above it. Unless there is an obstruction in the ureter, the pressure is elevated significantly only if the bladder is full. The kidney responds to the elevated pelvic pressure by reducing the renal blood flow and GFR, so as to slow the production of urine until the bladder is emptied and the pelvic pressure is reduced.

The physiologic responses of the kidney to the elevated pelvic pressure were investigated in relation to the disease “obstructive nephropathy”. The term obstructive nephropathy is used to describe the functional and pathologic changes in the kidney that result from obstruction to the flow of urine, raising renal pelvic, and eventually intrarenal, pressure to very high levels. Obstruction to the flow of urine can occur anywhere in the urinary tract and has many different causes. Significant obstruction to the flow of urine over a long period of time (a day to weeks) can result in renal failure and need surgical correction. Obstructive nephropathy is responsible for approximately 4% of the end-stage renal failure conditions in patients.

At the same time, obstruction of the urine flow and the associated increase of pelvic pressure for a short period of time (hours to a day) seems to be harmless. In “Reflux and Obstructive Nephropathy” James M. Gloor and Vicente E. Torres reported the recovery of renal function after the relief of complete unilateral ureteral obstruction of various durations. The recovery of the ipsilateral glomerular filtration rate after relief of a unilateral complete ureteral obstruction has been best studied in dogs and depends on the duration of the obstruction. Complete recovery always occurs after 1 week of obstruction, although the more prolonged the obstruction, the more prolonged the duration of renal dysfunction prior to total recovery. It takes from days to months of obstruction to induce permanent damage to the kidney. Based on this data, obstruction of urine outflow from one or two kidneys for several hours shall have no long-term effect on the kidneys.

The acute effect of elevated renal pelvis pressure on the function of the kidney was studied in animals. Hvistendahl et al described effects of the increased urine pressure on renal function in “Renal hemodynamic response to gradated ureter obstruction in the pig” (Nephron 1996; 74(1): 168-74). Hvistendahl reported that elevation of the ureteral pressure in steps of 10 mm Hg to a maximum of 80 mm Hg decreased ipsilateral (meaning blood flow to the kidney in the same side of the body in which the intervention was performed) Renal Blood flow (RBF) by 45% from 300 to 168 ml/min. Contralateral (the opposite side of the body or kidney without intervention) RBF did not change significantly. The mean arterial pressure was constant during the experimental procedures, suggesting that the decrease of RBF was due to a significant increase in ipsilateral renal vascular resistance. Concomitantly with these changes, ipsilateral GFR was reduced by 75% from 40 to 10 ml/min. In the contralateral kidney (kidney in the opposite side of the body), GFR was unchanged during the experiment.

Pedersen TS reported in similar findings in “Renal water and sodium handling during gradated unilateral ureter obstruction” (Scand J Urol Nephrol 2002; 36(3): 163-72). Peterson concluded that water reabsorbtion and sodium handling is progressively impaired with increasing renal pelvic (inside renal pelvis) pressure. The GFR and RBF are reduced in parallel. The study shows that both kidneys responds to ureteral obstruction of one kidney in unique and individual ways.

Lelarge et al reported the anecdotal clinical evidence supporting the invention in the “Acute unilateral renal failure and contralateral ureteral obstruction” (American Journal of Kidney Diseases. 20(3): 286-8, 1992 Sep). After obstetrical surgery woman developed an acute failure of one kidney. The ureter of the other kidney was ligated (ureter was clamped). Lelarge speculated that the kidney with the ligated (obstructed) ureter was somehow protected from injury.

Based on this physiologic data points it is reasonable to conclude that the elevation of the renal pelvic pressure to approximately 10 to 80 mm Hg for the duration of the high concentration of contrast of blood will protect the kidney from nephropathy by reducing the amount of blood that flows through the kidney and by the reduction of filtration (GFR).

To increase the pressure in the renal pelvis 701, a catheter 704 similar to a standard Foley catheter is placed in the bladder 703. The controller 114 is used to infuse fluid under pressure into the bladder and maintain bladder, thus ureteral and renal pelvic, pressures at the desired level. Catheter 704 can be equipped with an occlusion balloon, pressure sensing lumens and drainage lumens in addition to the fluid infusion lumen.

Alternatively, the catheter 704 can be placed in a ureter 702 or 705 if only one kidney needs to be protected (shut down). Laparoscopic procedure for the placement of a catheter in the ureter is described in U.S. Pat. No. 4,813,925, entitled Spiral Ureteral Stent. The balloon catheter system for the partial or complete ureteral occlusion is substantially the same as the design of the vascular catheter illustrated by FIG. 1 and FIG. 5. Partial occlusion of the ureter is more difficult to achieve than the occlusion of the bladder. At the same time it may be preferred because the contralateral kidney will be able to make urine during the procedure. If both kidneys are “turned off” with one of the methods described above, a common technique of hemodialysis of extracorporeal blood ultrafiltration can be used to replace renal function for the duration of treatment. A state of the art device such as the Prisma CRRT machine manufactured by Gambro AB (Stockholm, Sweden) can be used to remove excess fluid buildup in the body while the patient's kidneys are protected from high concentration of contrast in blood.

Fluid infused into the renal pelvis via the catheter to sustain elevated pressure can be colder than the body temperature. Cooling the kidney even by as little as 5-10 degrees below the overall body temperature can additionally reduce blood flow, GFR, metabolism in the kidney and protect it from the insult induced by contrast. Experience with renal transplantation confirms that the kidney is well protected by cold and recovers from it well when it is re-warmed. If continuous cooling is desired, the cooling fluid such as iced water or saline can be infused into the renal pelvis by an external pump that is part of the controller 114 and continuously drained out. The temperature of the cooling fluid can be controlled to avoid over-cooling. If the distension of the bladder or ureter by the elevated pressure becomes painful to the patient, a pain-reducing medication such as Novocain can be added to the fluid pumped into the renal pelvis or given systemically to the patient.

Common to all the embodiments, is that the renal blood flow and/or GFR of one or two kidneys are artificially reduced for the duration of the high concentration of radiocontrast in blood. This duration is typically equal to the time during which contrast is injected into the blood and stays mostly intravascular (dissolved in blood plasma). The kidney remains protected by “hibernation” for the duration of high concentration that is expected to last several hours while the contrast is redistributed from vascular compartment to the total body distribution volume.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for protecting a kidney in a mammalian patient from an insult comprising:
at least partially occluding at least one renal vein of the patient;
elevating a renal vein blood pressure, and reducing the renal vein blood pressure from the elevated blood pressure.

2. The method as in claim 1 wherein the blood pressure is elevated during a period of high concentration of contrast in blood of the patient.

3. The method as in claim 1 wherein the elevated blood pressure inhibits a renal function.

4. The method as in claim 3 wherein the inhibited renal function is a reduction in glomerular filtration rate (GFR).

5. The method is in claim 1 wherein insult is a contrast agent and the contrast agent may subject the kidney to radiocontrast nephropathy.

6. The method as in claim 1 wherein partially occluding the renal vein is accomplished by inserting a catheter tip with an inflatable balloon into the renal vein and inflating the balloon.

7. The method as in claim 6 wherein maintaining blood pressure further comprises sensing the renal vein pressure and adjusting the balloon in response to the sensed renal vein pressure.

8. The method as in claim 2 further comprises lodging the catheter in a branch of the renal vein distal of the balloon.

9. The method as in claim 1 further comprising injecting the contrast agent into a blood vessel of the patient.

10. The method as in claim 1 wherein the high concentration of contrast in blood occurs from injection of the contrast into a blood vessel to a fifty percent reduction in the concentration of contrast in blood from a peak contrast concentration.

11. The method as in claim 1 wherein the renal vein blood pressure is elevated to a range of 30 to 60 mmHg.

12. The method as in claim 1 wherein the renal vein pressure is elevated to a range of 30 to 60 mmHg above a baseline venous pressure of The patient.

13. The method as in claim 3 wherein a balloon size is adjusted based on a sensed renal vein pressure.

14. The method for minimizing radiocontrast nephropathy in a mammalian patient comprising:
at least partially occluding at least one renal vein of the patient, and
elevating a renal vein blood pressure during a period during a period coinciding with an injection of contrast in blood of the patient.

15. The method as in claim 14 wherein the blood pressure is elevated during a period of high concentration of the contrast in the blood of the patient.

16. The method as in claim 14 wherein the elevated blood pressure inhibits a renal function.

17. The method as in claim 16 wherein the inhibited renal function is a reduction in glomerular filtration rate (GFR).

18. The method as in claim 14 wherein the renal pressure is elevated prior to the injection of the contrast agent.

19. The method as in claim 14 wherein partially occluding the renal vein is accomplished by inserting an expandable catheter tip.

20. The method as in claim 19 wherein the catheter tip further comprises an inflatable balloon, which is inflated after being positioned in the renal vein.

21. The method as in claim 20 wherein maintaining blood pressure further comprises sensing the renal vein pressure and adjusting the balloon in response to the sensed renal vein pressure.

22. The method as in claim 19 further comprises lodging the catheter tip in a branch of the renal vein distal of the balloon.

23. The method as in claim 14 further comprising injecting the contrast agent into a blood vessel of the patient.

24. The method as in claim 23 further wherein the period of contrast occurs from injection of the contrast into a blood vessel to a fifty percent reduction in the concentration of the contrast in the blood from a peak contrast concentration.

25. The method as in claim 14 wherein the renal vein blood pressure is elevated to a range of 30 to 60 mmHg.

26. The method as in claim 14 wherein the renal vein pressure is elevated to a range of 30 to 60 mmHg above a baseline venous pressure of the patient.

27. The method as in claim 20 wherein a balloon size is adjusted based on a sensed renal vein pressure.

28. The system for treating radiocontrast nephropathy in a mammalian patient comprising:
a renal catheter further comprising a distal tip section having a renal vein occlusion device and a renal vein pressure detector, and a proximal section external of the patient when the distal tip section is positioned in a renal vein, and
an actuator for the renal vein occlusion device and connectable to the proximal section of the renal catheter, wherein said actuator controls the renal vein occlusion device.

29. The system as in claim 28 further comprising a controller for the actuator wherein said controller monitors the renal vein pressure based on signals from the pressure detector and actuates the occlusion device in response to the renal vein pressure

30. The system as in claim 28 wherein the occlusion device is an expandable device at a distal section of the catheter.

31. The system as in claim 30 wherein the expandable device is positionable in a renal artery leading to the at least one kidney.

32. The system for artificially protecting a kidney during a renal insult in a mammalian patient comprising:
means for at least partially occluding at least one renal vein of the patient, and
means for controlling an increase in renal vein blood pressure during a period corresponding to the insult.

33. The system as in claim 32 wherein the renal insult is a radiocontrast infusion and the period corresponding to the insult is a period of high concentration of contrast in blood of the patient.

34. The system as in claim 32 wherein the renal insult is a surgical procedure.

35. The system as in claim 32 wherein the renal insult is a hypotension.

36. The system as in claim 32 wherein the means for at least partially occluding further comprises a catheter having an expandable device at a distal section of the catheter.

37. The system as in claim 36 wherein the expandable device is positionable in a renal artery of the at least one kidney.

38. The system for artificially protecting a kidney during a renal insult in a mammalian patient comprising:
a renal catheter further comprising a distal tip section having a renal vein occlusion device and a renal vein pressure detector, and a proximal section external of the patient when the distal tip section is positioned in a renal vein, and
an actuator for the renal vein occlusion device and connectable to the proximal section of the renal catheter, wherein said actuator controls the renal vein occlusion device.

39. The system as in claim 38 further comprising a controller for the actuator wherein said controller monitors the renal vein pressure based on signals from the pressure detector and actuates the occlusion device in response to the renal vein pressure

40. The system as in claim 38 wherein the occlusion device is an expandable device at a distal section of the catheter.

41. The system as in claim 38 wherein the expandable device is positionable in a renal artery leading to the at least one kidney.

42. The system as in claim 38 wherein the renal insult is a radiocontrast infusion.

43. The system as in claim 38 wherein the renal insult is a surgical procedure.

44. The system as in claim 38 wherein the renal insult is a hypotension.

45. The system as in claim 38 wherein the means for at least partially occluding further comprises a catheter having an expandable device at a distal section of the catheter.

46. The system as in claim 45 wherein the expandable device is positionable in a renal artery of the at least one kidney.
